SEQUENCE LISTING (1 sequence)

SEQ ID NO: 1 - protein, 32 aa, Homo sapiens
SDGDWICPDKKCGNVNFARRTSCNRCGREKTT

(19) United States
(12) Patent Application Publication
(10) Pub. No.: US 2025/0179129 A1
(43) Pub. Date: Jun. 5, 2025

(54) ZINC FINGER PEPTIDES, PEPTIDE ARRAYS, AND METHODS OF USE THEREOF

(71) Applicant: The Regents of the University of California, Oakland, CA (US)

(72) Inventors: Kevin D. Corbett, San Diego, CA (US); Eugene Yeo, La Jolla, CA (US); Qishan Liang, La Jolla, CA (US)

(21) Appl. No.: 18/840,523

(22) PCT Filed: Feb. 23, 2023
(86) PCT No.: PCT/US2023/063128

§ 371 Date: Aug. 22, 2024

Related U.S. Application Data
(60) Provisional application No. 63/313,029, filed on Feb. 23, 2022.

(51) Int. Cl. C07K 14/47; A61K 38/00; C07K 19/00

(52) U.S. Cl. CPC C07K 14/4702; C07K 19/00; A61K 38/00

(57) ABSTRACT

Provided herein are zinc finger peptides comprising the amino acid sequence of SEQ ID NO: 1 (SDGDWICPDKKCGN-VNFARRTSCNRCGREKTT) and at least one substitution compared to SEQ ID NO: 1, wherein the zinc finger peptide is an engineered zinc finger peptide.

### CROSS-REFERENCE TO RELATED APPLICATIONS

This application claims priority to U.S. Provisional Patent Application No. 63/313,029, filed on Feb. 23, 2022. The disclosure of the prior application is considered part of the disclosure of this application, and is incorporated herein by reference in its entirety.

### STATEMENT REGARDING FEDERAL FUNDING

This invention was made with government support under grants HG004659 and GM128464 awarded by the National Institutes of Health. The government has certain rights in the invention.

### SEQUENCE LISTING

This application contains a Sequence Listing that has been submitted electronically as an ASCII text file named Sequence_Listing. The ASCII text file, created on Feb. 23, 2023, is 1,939 bytes in size. The material in the ASCII text file is hereby incorporated by reference in its entirety.

### TECHNICAL FIELD

The present disclosure relates to zinc finger peptides, peptide arrays, and method of use thereof (e.g. targeting RNA sequences). Optionally, the zinc finger peptides can be fused to effector proteins that, for example, facilitate nucleic acid degradation, nucleic acid splicing control, or nucleic acid translation of the target nucleic acid sequence.

### BACKGROUND

Existing nucleic acid-targeting tools (e.g. RNA-targeting tools) for sequence-specific manipulation include anti-sense oligos (ASOs), designer PUF (Pumilio and FBF homology protein) proteins, and CRISPR (Clustered Regularly Interspaced Short Palindromic Repeats)/Cas systems. However, there are significant limitations to each of the current tools. ASOs are usually not available for most RNA manipulations other than gene silencing. Designer proteins, such as PUFs, possess low RNA recognition efficiency and it remains challenging to target RNA sequences greater than 8-nucleotides (nt) in length. The bulkiness of the Cas protein (Cas13d: average 930 amino acids) complicates transgene delivery and its bacterial origin poses immunogenicity challenges. There is a need improved methods and compositions used to target nucleic acids, particularly RNA.

### SUMMARY

Targeting and manipulating endogenous RNAs in a sequence-specific manner is important for developing RNA-targeting therapeutics. RNA-recognizing zinc fingers are excellent candidates as designer proteins to expand the RNA-targeting toolbox due to their compact size (˜3kDa each) and modular sequence recognition. Described herein are zinc finger peptides that include the amino acid sequence of SEQ ID NO: 1 (SDGDWICPDKKCGNVNFARRTSCNRCGREKTT) and at least one substitution compared to SEQ ID NO: 1. In some cases, the zinc finger peptide is an engineered zinc finger peptide. In some cases, the zinc finger peptide has i) a substitution of N24R compared to SEQ ID NO: 1, and/or ii) a substitution of N24H compared to SEQ ID NO: 1, and/or iii) substitutions of N14D and N24R compared to SEQ ID NO: 1, and/or iv) substitutions of N14D and N24H compared to SEQ ID NO: 1, and/or v) substitutions of N14R and N24R compared to SEQ ID NO: 1, and/or vi) substitutions of N14R and N24H compared to SEQ ID NO: 1, and/or vii) substitutions of N14H and N24R compared to SEQ ID NO: 1, and/or viii) substitutions of N14H and N24H compared to SEQ ID NO: 1, and/or ix) substitutions of N14Q and N24R compared to SEQ ID NO: 1, and/or x) substitutions of N14Q and N24H compared to SEQ ID NO: 1, and/or xi) substitutions of N14E and N24R compared to SEQ ID NO: 1, and/or xii) substitutions of N14S and N24R compared to SEQ ID NO: 1, and/or xiii) substitutions of N14E and N24H compared to SEQ ID NO: 1.

Also described herein are zinc finger peptide arrays that include a) 2-30 zinc finger peptides with different preferred RNA binding motifs, and b) a nucleotide spacer between the 2-30 zinc finger peptides. In some cases, the zinc finger peptide array comprises 6 zinc finger peptides. In some cases, the nucleotide spacer is between 1 and 10 nucleotides in length.

Also described herein are methods of targeting nucleic acid sequences by exposing an RNA sample to any of the zinc finger peptides or zinc finger peptide arrays described here. In some cases, the zinc finger peptide array is fused to an effector protein. In some cases, the effector protein facilitate a function selected from nucleic acid degradation, nucleic acid splicing control, and nucleic acid translation

Also described herein are methods of treating a G4-associated disease in a patient in need thereof including administering a therapeutically effective amount of any of the zinc finger peptides or any of the zinc finger peptide arrays described herein. In some cases, the G4-associated disease is selected from the group consisting of amyotrophic lateral sclerosis (ALS) and frontotemporal dementia (FTD).

In any of the zinc finger peptides, zinc finger peptide arrays, or methods described herein, the zinc finger peptide binds to RNA. In some cases, the zinc finger peptide has a preferred RNA binding motif that is not a GGU RNA sequence. In some cases, the zinc finger peptide has a preferred RNA binding motif that is a GGG RNA sequence. In some cases, the preferred RNA binding motif is determined with a fluorescent polarization assay or an electrophoretic mobility shift assay.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1C show an exemplary single-strand zinc finger (ZnF) array interacting with a single stranded RNA. FIG. 1A is a schematic of a single stranded RNA molecule where different colored boxes of the single stranded RNA represent different nucleotides. FIG. 1B is an exemplary single-strand zinc finger (ZnF) array (ZRANB2-ZNF1) interacting with a single stranded RNA at a molecular level, with key amino acids indicated. FIG. 1C shows exemplary binding motifs of ZnF1 and ZnF2.

FIGS. 2A-2C show an example of systematic mutagenesis and high-throughput purification of ZRANB2 zinc fingers. FIG. 2A shows an exemplary protocol for systematic mutagenesis of ZRANB2 zinc finger 1 (ZnF1). Key residues are indicated with a grey box. FIG. 2B is an exemplary protein expression construct. His=polyhistidine tag; MBP=maltose-binding peptide motif; SBP=streptavidin binding peptide motif; ZnF-WT=zinc finger wild-type motif; ZnF-Mt=zinc finger mutant motif. FIG. 2C is an exemplary protein purification protocol.

FIGS. 3A-3D show results from the characterization of RNA-binding profile of each mutant zinc finger using an RNA-bind-n-seq assay. FIG. 3A shows a streptavidin coated magnetic bead interacting with a mutant protein construct through streptavidin bind peptide motif. FIG. 3B showed mutant or wild-type zinc finger proteins (ZnF protein) interacting with random RNAs. FIG. 3C shows an exemplary sequencing construct. FIG. 3D shows fold-enrichment of RNA binding sequences.

FIG. 4 shows determination of core binding motifs for the zinc-finger wild-type, zinc finger mutant SNF-RBM5, and a dead mutant.

FIG. 5 shows determination of core binding motifs for additional zinc-finger mutants.

FIGS. 6A-B show a series of graphs of the hydrogen-binding (H-bind) intensity score of various zinc-finger mutants and wild-type with either GGU RNA or GGG RNA.

### DETAILED DESCRIPTION

Targeting and manipulating endogenous RNAs in a sequence-specific manner is important for both understanding RNA biology and developing RNA-targeting therapeutics. RNA-recognizing zinc fingers (ZnFs) are excellent candidates as designer proteins due to their compact size (˜3 kDa each) and modular sequence recognition. A zinc finger in ZRANB2 recognizes a single-strand RNA containing a GGU motif with micromolar affinity. Although structures of ZRANB2 ZnF in complex with the RNA have been solved (See, for example, Loughlin, F. E. et al. Proc. Natl. Acad. Sci. 106(14):5581-6 (2009)), the rules that govern its sequence-specific recognition are still unclear.

Described herein are methods and compositions including a recombinant zinc finger peptide comprising the amino acid sequence of SEQ ID NO: 1 (SDGDWICPDKKCGNVNFARRTSCNRCGREKTT) and at least one substitution compared to SEQ ID NO: 1. Various non-limiting aspects of these methods and compositions are described herein, and can be used in any combination without limitation.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelle biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an [symbol] -L-ribo configuration (a diastereomer of LNA), 2′-amino-LNA having a 2′-amino functionalization, and 2′-amino- [symbol] -LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single-or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. In some embodiments, nucleic acid modifications can also include a blocking modification comprising a 3′ end modification (e.g., a 3′ dideoxy C (3′ddC), 3′ddG, 3′ddA, 3′ddT, 3′ inverted dT, 3′ C3 spacer, 3′ amino, 3′ biotinylation, or 3′ phosphorylation). The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

As used herein, the term “recombinant” refers to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

### Zinc-Finger Peptides

Zinc finger proteins (ZFPs) are proteins that can bind to DNA or RNA in a sequence-specific manner. Zinc fingers were first identified in the transcription factor TFIIIA from the oocytes of the African clawed toad, Xenopus laevis. A “zinc finger” or “zinc finger domain” is a relatively small polypeptide domain comprising approximately 30 amino acids, which folds to form a secondary structure including an α-helix adjacent an antiparallel b-sheet (known as a bba-fold). The fold is stabilized by the co-ordination of a zinc ion between four largely invariant (depending on zinc finger framework type) Cys and/or His residues, as described further below. Natural zinc finger domains have been well studied and described in the literature, see for example, Miller et al., (1985) EMBO J. 4:1609-1614; Berg (1988) Proc. Natl. Acad. Sci. USA 85:99-102; and Lee et al., (1989) Science 245:635-637.

The most widely represented class of ZFPs is also known as C2H2 ZFPs, wherein most zinc finger proteins have conserved cysteine and histidine residues that tetrahedrally-coordinate the single zinc atom in each finger domain. Additional classes of ZFPs have also been suggested. (e.g., Cys-Cys-His-Cys (C3H) ZPFs (e.g., see Jiang et al. (1996) J. Biol. Chem. 271:10723-10730)).

The zinc-coordinating sequences of the most widely represented class contain two cysteines and two histidines with particular spacings. For example, zinc fingers found in the yeast protein ADRI, the human male associated protein ZFY, the HIV enhancer protein and the Xenopus protein Xfin have been solved by high resolution NMR methods (Kochoyan, et al., Biochemistry, 30:3371-3386, 1991; Omichinski, et al., Biochemistry, 29:9324-9334, 1990; Lee, et al., Science, 245:635-637, 1989). The folded structure of each finger contains an antiparallel β-turn, a finger tip region and a short amphipathic α-helix. The metal coordinating ligands bind to the Zn ion and, in the case of zif268 zinc fingers, the short amphipathic α-helix binds in the major groove of DNA. In addition, the conserved hydrophobic amino acids and zinc coordination by the cysteine and histidine residues stabilize the structure of the individual finger domain.

In some embodiments, the folding of a C2H2 ZFP into the proper finger structure can be entirely disrupted by exchange of the C2H2 ligand amino acids (e.g., see, Miura et al. (1998) Biochim. Biophys. Acta 1384:171-179). Furthermore, metal binding specificity of peptides based on the C2H2 consensus sequence can be altered (e.g., see, Krizek et al. (1993) Inorg. Chem. 32:937-940; Merkle et al. (1991) J. Am Chem. Soc. 113:5450-5451).

In some embodiments, zinc finger domains are involved not only in DNA recognition, but also in RNA binding and in protein-protein binding. In some embodiments, a zinc finger domain typically recognizes and binds to a nucleic acid triplet, or an overlapping quadruplet (as explained below), in a double-stranded DNA target sequence. However, zinc fingers are also known to bind RNA and proteins (Clemens, K. R. et al. (1993) Science 260:530-533; Bogenhagen, D. F. (1993) Mol. Cell. Biol. 13:5149-5158; Searles, M. A.

et al. (2000) J. Mol. Biol. 301:47-60; Mackay, J. P. & Crossley, M. (1998) Trends Biochem. Sci. 23:1-4). In some cases, the zinc finger peptides described herein recognize (or bind) RNA.

Zinc finger domains are compact protein domains composed of an α-helix and β-sheet held together by a single zinc ion. In some embodiments, tandem arrays of zinc fingers are commonly used to recognize specific DNA sequences through insertion of α-helices into the DNA major groove. In some embodiments, DNA-binding fingers can be designed by using the structures of DNA-protein complexes as guides. In some embodiments, zinc finger proteins can also bind specific RNA sites. In some embodiments, zinc finger proteins can bind RNA via contacts with amino acid side chains in the α-helical portion of the zinc finger. In some embodiments, in vitro selection and recombination techniques have isolated single zinc fingers that bind complex RNA structures with high affinity and specificity, wherein these zinc fingers may be specifically engineered to modify the function of cellular or viral RNA. For example, TFIIIA is a nine-finger protein that binds specifically to both DNA and RNA but uses different sets of fingers and different features of the major groove in each case. In some embodiments, other zinc fingers can bind to DNA-RNA hybrids.

Zinc finger proteins generally contain strings or chains of zinc finger domains (or modules). Thus, a natural zinc finger protein may include two or more zinc finger domains, which may be directly adjacent one another, e.g. separated by a short (canonical) or canonical-like linker sequence; or a longer, flexible or structured polypeptide sequence. Adjacent zinc finger domains linked by short canonical or canonical-like linker sequences of 5, 6 to 7 amino acids are expected to bind to contiguous nucleic acid sequences, i.e. they typically bind to adjacent trinucleotides/triplets; or protein structures. In some cases, cross-binding may also occur between adjacent zinc fingers and their respective target triplets, which helps to strengthen or enhance the recognition of the target sequence, and leads to the binding of overlapping quadruplet sequences (Isalan et al., (1997) Proc. Natl. Acad. Sci. USA, 94:5617-5621). By comparison, distant zinc finger domains within the same poly-zinc finger protein may recognize (or bind to) non-contiguous nucleic acid sequences or even to different molecules (e.g. protein rather than nucleic acid). Indeed, naturally occurring zinc finger-containing proteins may include both zinc finger domains for binding to protein structures as well as zinc finger domains for binding to nucleic acid sequences.

Zinc finger peptides have a binding motif to recognize their RNA target. In some embodiments, engineering can alter the zinc finger peptide's preferred binding motif. In some cases, any of the zinc finger peptides described herein have a preferred RNA binding motif that is not a GGU RNA sequence. In some cases, the zinc finger peptide has a preferred RNA binding motif that is a GGG RNA sequence. In some cases, zinc finger peptide binds to a 5′-GGGGCC-3′ nucleic acid repeat sequence. Binding motifs can be identified using a variety of analyses. Example 1 describes using a kpLogo analysis to identify the binding motif to zinc finger Ran-binding domain-containing protein 2 (ZRANB2). In addition, the RNA binding motif can be determined with a fluorescent polarization assay, an electrophoretic mobility shift assay, or any other assay that determines binding affinity of a protein to an RNA molecule.

Provided herein are zinc finger peptides comprising the amino acid sequence of SEQ ID NO: 1 (SDGDWICPDKKCGNVNFARRTSCNRCGREKTT) and at least one substitution compared to SEQ ID NO: 1. In some cases, the zinc finger peptide is an engineered zinc finger peptide. In some embodiments, zinc finger peptides can include a mutation or modification compared to the zinc-finger peptide of SEQ ID NO: 1. Substitutions can include the substitution of amino acid R at amino acid position 42 for amino acid N (N42R) compared to SEQ ID NO: 1, and/or a substitution of N24H compared to SEQ ID NO: 1, and/or substitutions of N14D and N24R compared to SEQ ID NO: 1, and/or substitutions of N14D and N24H compared to SEQ ID NO: 1, and/or substitutions of N14R and N24R compared to SEQ ID NO: 1, and/or substitutions of N14R and N24H compared to SEQ ID NO: 1, and/or substitutions of N14H and N24R compared to SEQ ID NO: 1, and/or substitutions of N14H and N24H compared to SEQ ID NO: 1, and/or substitutions of N14Q and N24R compared to SEQ ID NO: 1, and/or substitutions of N14Q and N24H compared to SEQ ID NO: 1, and/or substitutions of N14E and N24R compared to SEQ ID NO: 1, and/or substitutions of N14S and N24R compared to SEQ ID NO: 1, and/or substitutions of N14E and N24H compared to SEQ ID NO: 1.

### Zinc Finger Peptide Arrays

As used herein, a “zinc finger peptide array” refers to a plurality of zinc finger domains linked into tandem arrays that allow the plurality of proteins to recognize extended nucleic acid sequences. Multiple zinc finger peptides can be linked to generate zinc finger arrays of any desired length. For example, two, three, four, five, or up to thirty zinc finger peptides can be linked to generate zinc finger arrays. In some cases, the zinc finger peptide array comprises six zinc finger peptides.

In zinc finger peptide arrays of the present invention, adjacent zinc finger domains are joined to one another by ‘linker sequences’ that may be canonical, canonical-like, flexible or structured, as described, for example, in WO 01/53480 (Moore et al., (2001) Proc. Natl. Acad. Sci. USA 98:1437-1441). Generally, a natural zinc finger linker sequence lacks secondary structure in the free form of the peptide. However, when the protein is bound to its target site a canonical linker is typically in an extended, linear conformation, and amino acid side chains within the linker may form local interactions with the adjacent nucleic acid. In a tandem array of zinc finger domains, the linker sequence is the amino acid sequence that lies between the last residue of the a-helix in an N-terminal zinc finger and the first residue of the b-sheet in the next (i.e. C-terminal adjacent) zinc finger. In some cases, the ‘linker sequences’ are nucleotide spacers between the two to thirty zinc finger peptides. In some cases, the nucleotide spacer is between about 1 and about 10 nucleotides in length (e.g., between about 1 and about 8 nucleotides, between about 1 and about 6 nucleotides, between about 1 and about 4 nucleotides, between about 2 and about 10 nucleotides, between about 2 and about 8 nucleotides, between about 2 and about 6 nucleotides, between about 2 and about 4 nucleotides, between about 3 and about 10 nucleotides, between about 3 and about 8 nucleotides, between about 3 and about 6 nucleotides, between about 3 and about 4 nucleotides, between about 5 and about 10 nucleotides, between about 5 and about 8 nucleotides, between about 5 and about 6 nucleotides, or between about 8 and about 10 nucleotides).

### Methods of Targeting a Nucleic Acid Sequence

Provided herein are methods of targeting nucleic acid sequences that include exposing an RNA sample to a zinc finger peptide array, wherein the zinc finger peptide array comprises: a) 2-30 zinc finger peptides with different preferred RNA binding motifs, and b) a nucleotide spacer sequence between the 2-30 zinc finger peptides. Any of the zinc finger peptides, engineered zinc finger peptides, or zinc finger peptide arrays described herein can be used to target nucleic acid sequences (e.g., motif binding sequences). In some embodiments, the methods can include exposing an RNA sample to any of the zinc finger peptides or zinc finger peptide arrays described herein.

In some cases, a zinc finger peptide includes a preferred RNA binding motif, wherein the preferred RNA binding motif recognizes a target RNA. In some cases, the preferred RNA binding motif can recognize a target RNA by hybridizing to the target RNA. In some cases, the preferred RNA binding motif comprises a sequence that is complementary to the target RNA.

In some cases, the preferred RNA binding motif recognizes a target RNA that includes a GGG RNA sequence. In some cases, the preferred RNA binding motif does not recognize a GGU RNA sequence. In some cases, the preferred RNA binding motif selectively binds to a mutant RNA sequence but not wild-type transcripts. In some cases, the preferred RNA binding motif binds to a 5′-GGGGCC-3′ nucleic acid repeat sequence.

In some cases, the preferred RNA binding motif can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (IncRNA), microRNA (miRNA), piwi-interacting RNA (piRNA), small interfering RNA (siRNA), short hairpin RNA (shRNA), retrotransposon RNA, viral genome RNA, or viral noncoding RNA. In some cases, a target RNA can be an RNA involved in pathogenesis of conditions such as repeat expansion diseases. In some cases, a target RNA can be an RNA involved in a G4-associated disease (e.g., amyotrophic lateral sclerosis (ALS) or frontotemporal dementia (FTD)).

In some cases, the zinc finger peptide or zinc finger peptide array is fused to an effector protein. As used herein, an “effector protein” refers to a protein that selectively binds to a binding sequence, wherein the effector protein can facilitate a function or perform an action to the binding sequence (e.g., the RNA binding sequence). In some cases, the effector protein can facilitate a function selected from nucleic acid degradation, nucleic acid splicing control, and nucleic acid translation. For example, the zinc finger peptide or zinc finger peptide array can bind to its' binding target (e.g., binding RNA sequence) and cause a degradation of the RNA transcript, thereby preventing translation of the peptide encoded by the RNA transcript. In some cases, the effector protein can include a transcriptional activator. In some cases, the effector protein can include a transcriptional repressor. In some cases, the effector protein can include a methylation domain. In some cases, the effector protein can include a nuclease. In some cases, the effector protein can include a nuclease, wherein the nuclease causes the degradation of the target RNA transcript.

### Methods of Treating Neurological Disorders

Any of the zinc finger peptides, engineered zinc finger peptides, or zinc finger peptide arrays can be used to treat a G4-associated disease in a patient in need thereof.

Current knowledge of neurological disorders suggests that they can be caused by many different factors, including (but not limited to): inherited genetic abnormalities, problems in the immune system, injury to the brain or nervous system, or diabetes. One known cause of neurological disorder is a genetic abnormality leading to the pathological expansion of nucleic acid repeats sequences. As used herein, “repetitive RNA” or “repeat sequence” can refer to short or long patterns of nucleic acids (e.g., DNA or RNA) that occur in multiple copies throughout the genome. In some embodiments, these repeated sequences are necessary for maintaining important genome structures such as telomeres or centromeres. In some cases, repeated sequences can be important for cellular functioning and genome maintenance, while other repetitive sequences can be harmful. In some cases, repetitive RNA sequences are associated with diseases.

G4 repeats (e.g., GGGGCC repeats) in the C90RF72 gene in amyotrophic lateral sclerosis (ALS) or frontotemporal dementia (FTD) (DeJesus-Hernandez et al. (2011), Neuron, 72:245-56). The GGGGCC repeat expansion in C90RF72 appears to cause errors in splicing transcript formation that leads to an overall downregulation of correctly-spliced C90RF72 expression. Moreover, there is aberrant Repeat-Associated Non-AUG (RAN) dependent translation of the expanded C90RF72 transcript, leading to toxic peptide production that is thought to be important in the pathogenesis of ALS. This is also true in another repeat-expansion disease, Fragile X-Associated Tremor/Ataxia Syndrome (FXTAS) that is associated with CGG repeats and RAN translation toxicity (Kong et al., (2017) Frontiers in Cellular Neuroscience, 11, 128).

Amyotrophic lateral sclerosis (ALS) is a devastating neurological disease that belongs to the wider group of disorders known as ‘motor neuron diseases’, which are characterized by the gradual and progressive deterioration (degeneration) of the nerve cells (motor neurons) that control muscle movements. The disease, which is the most common motor neuron disease among adults, affects about 1 in 50,000 people and is currently without a cure. ALS tends to appear in mid-life (between the ages of 40 and 60), and affects men more frequently than women. In most cases, it appears to occur at random with no family history of the disease.

Frontotemporal dementia (FTD) is a relatively rare form of dementia, which occurs when nerve cells in the frontal and/or temporal lobes of the brain die, and the pathways that connect the lobes change as a result. Some of the chemical messengers that transmit signals between nerve cells are also lost. Over time, as more and more nerve cells die, the brain tissue in the frontal and temporal lobes shrinks, resulting in changes in personality and behavior, and difficulties with language. These symptoms are initially different from the memory loss often associated with more common types of dementia, such as Alzheimer's disease, but as the disease progresses more of the brain becomes damaged and symptoms are often similar to those of the later stages of Alzheimer's disease. About 10 to 20% of people with FTD develop a motor neuron disorder.

To date, treatments for these and similar diseases, have generally focused on trying to control the symptoms of rather than the causes of illness. The U.S. Food and Drug Administration (FDA) has approved the drugs riluzole (Rilutek™) and edaravone (Radicava™) to treat ALS. Riluzole is believed to reduce damage to motor neurons by decreasing levels of glutamate, which transports messages between nerve cells and motor neurons. Clinical trials in people with ALS showed that riluzole may prolong survival by a few months, but does not reverse the damage already done to motor neurons. Edaravone has also been shown to slow the decline in clinical assessment of daily functioning in persons with ALS. Other medications often prescribed to treat immediate symptoms of the disease include drugs such as baclofen or diazepam to help control spasticity; gabapentin to help control pain; and trihexyphenidyl or amitriptyline to help patients swallow. There are no recognized treatments to specifically target FTD and any treatments focus on the symptoms; for example, patients may be prescribed behavioral modification drugs. In some cases, patients previously diagnoses with FTD may be prescribed drugs that are used to treat Alzheimer's disease.

The hexanucleotide repeat expansions in the C9orf72 gene were discovered to be a common cause of both frontotemporal dementia (FTD) and amyotrophic lateral sclerosis (ALS). These two disorders have been known to occur within the same family, and even within the same person, but C9orf72 expansions provided an incontrovertible molecular link. Although frequency varies geographically, expansions in C9orf72 account for a high proportion of patients with familial FTD and ALS in most countries.

The C9orf72 gene on chromosome 9 has three transcription variants, and two possible protein isoforms. The hexanucleotide GGGGCC (G4C2) repeat region is located either in the promoter or in intron 1 of the gene, depending on the transcript variant. The normal repeat size is variable, with more than 90% of the European population having between two and ten G4C2 repeat units. Larger repeats are less common, and repeats of more than 30 units in length are an important, albeit rare, finding in healthy populations. Repeat expansions typically seen in ALS and/or FTD patients are far larger than this normal range, consisting of at least several hundred or, more often, thousands of repeats (Rohrer et al., Lancet Neurol. 2015 Mar;14 (3): 291-301).

Described herein are zinc finger peptides and zinc finger peptide arrays that can target G4-sequences (e.g., the GGGCC expansion) associated with ALS and/or FTD.

Any of the zinc finger peptides and zinc finger peptide arrays can be part of a pharmaceutical composition of which a therapeutically effective amount can be administered to treat ALS or FTD in a patient in need thereof.

Provided herein are methods of treating a G4-associated disease in a patient in need thereof, the method including administering a therapeutically effective amount of any one of the zinc finger peptides or any one of the zinc finger peptide arrays described herein. In some cases, the methods can include the administration of pharmaceutical compositions and formulations including vectors delivering a therapeutically effective amount of any one of the zinc finger peptides or any one of the zinc finger peptide arrays described herein.

### Vectors

In some embodiments, the zinc finger proteins or zinc finger arrays described herein (e.g., mutated, modified, and/or recombinant zinc finger proteins and/or zinc finger arrays) are packaged in one or more vectors. In some embodiments, the vector is a viral vector. In some embodiments, the viral vector includes a sequence isolated or derived from a retrovirus. In some embodiments, the viral vector includes a sequence isolated or derived from a lentivirus. In some embodiments, the viral vector includes a sequence isolated or derived from an adenovirus. In some embodiments, the viral vector includes a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the viral vector includes a sequence isolated or derived from an adeno-associated virus (AAV). In some embodiments, the viral vector includes an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, AAV12, AAV.rh32/33, AAV.rh43, AAV.rh64R1, and any combinations or equivalents thereof. In some embodiments, the viral vector is replication incompetent. In some embodiments, the viral vector is isolated or recombinant (rAAV). In some embodiments, the viral vector is self-complementary (scAAV). In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb.

In some embodiments, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. As used herein, a “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3 (2): 132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVsM) vector, a modified sooty mangabey simian immunodeficiency virus (SIVsM) vector, a African green monkey simian immunodeficiency virus (SIVAGm) vector, a modified African green monkey simian immunodeficiency virus (SIVAGm) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments, the vector can be introduced into any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. In some embodiments, the vector can be delivered into the cytoplasm of a cell. In some embodiments, the vector can be delivered into the cell by chemical transfection, non-chemical transfection, particle-based transfection, or viral transfection. In some embodiments, the vector can be delivered with a transfection reagent.

### Pharmaceutical Compositions

In some embodiments, the vectors encoding the mutated zinc finger proteins described herein are administered to a subject via a pharmaceutical composition. In some embodiments, the pharmaceutical compositions are formulated with a pharmaceutically acceptable carrier. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The pharmaceutical composition can be administered alone or as a component of a pharmaceutical formulation. The compounds may be formulated for administration, in any convenient way for use in human or veterinary medicine. The compositions may conveniently be presented in unit dosage form and may be prepared by any methods well known in the art of pharmacy. The amount of active ingredient which can be combined with a carrier material to produce a single dosage form can vary depending upon the host being treated, the particular mode of administration. The amount of active ingredient which can be combined with a carrier material to produce a single dosage form will generally be that amount of the compound which produces a therapeutic effect.

Pharmaceutical compositions described herein can be prepared according to any method known to the art for the manufacture of pharmaceuticals. Such compositions can contain, for example, preserving agents. A composition can be admixtured with nontoxic pharmaceutically acceptable excipients which are suitable for manufacture. Compositions may comprise one or more diluents, emulsifiers, preservatives, buffers, excipients, etc. and may be provided in such forms as liquids, powders, emulsions, lyophilized powders, controlled release formulations, on patches, in implants, etc. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences as described herein. As an example of an injectable oil vehicle, see Minto (1997) J. Pharmacol. Exp. Ther. 281:93-102.

Pharmaceutical compositions can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as egg or soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

In some embodiments, the pharmaceutical compositions can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compositions can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono-or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/ml protein, about 15 mg/ml sucrose, about 19 mg/ml NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

### Administration

In some embodiments, the vectors encoding the mutated zinc finger proteins described herein are administered to a subject via a pharmaceutical composition. In some embodiments, the administration of the pharmaceutical composition comprises intrastriatal administration. In some embodiments, the administration of the pharmaceutical composition comprises ocular, oral, parenteral, bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, transcutaneous, subcutaneous, sublingual, tracheal (e.g., by intratracheal instillation), vaginal, or vitreal. In some embodiments, the pharmaceutical composition is administered by enteral administration, intraperitoneal administration, intravenous administration, intramuscular administration, subcutaneous administration, intracutaneous administration, oral administration, intranasal administration, intrapulmonary administration, intrarectal administration, intrastriatal administration or a telemetry controlled external or implanted infusion pump.

A patient can include a human, a non-human primate, pets (e.g., cats and dogs), and farm animals (e.g., cows, horses, chickens, pigs, etc.).

In some cases, the pharmaceutical composition is for use in a medicine.

### Method of Making Zinc-Finger Peptides or Zinc Finger Peptide Arrays

In some cases, the zinc-finger peptide, or engineered zinc finger peptide, can expressed inside a host cell. As used herein, the term “expression” refers to the process by which polynucleotides are transcribed into mRNA and/or the process by which the transcribed mRNA is subsequently translated into peptides, polypeptides, or proteins. As used herein, a “host cell” can refer to either a prokaryotic or eukaryotic cell, optionally obtained from a subject or a commercially available source. Appropriate host cell can be used to express the construct encoding the zinc-finger peptide, or the engineered zinc-finger peptide. Non-limiting examples include bacteria (such as Escherichia coli) and yeast (such as Saccharomyces cerevisiae or Pichia pastoris).

The construct used to express the zinc peptide or engineered zinc-finger peptide can be exogenous to the host cell. As used herein, the term “exogenous” refers to any material introduced from or originating from outside a cell, a tissue or an organism that is not produced by or does not originate from the same cell, tissue, or organism in which it is being introduced.

The construct used to express the zinc peptide or engineered zinc-finger peptide can be delivered to the host cell using any known method in the art. As used herein, “delivering”, “gene delivery”, “gene transfer”, “transducing” can refer to the introduction of an exogenous polynucleotide into a host cell, irrespective of the method used for the introduction. Such methods include a variety of well-known techniques. Non-limiting examples include vector-mediated gene transfer (e.g., transduction, viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some cases, the construct is a DNA construct encoding a zinc-finger peptide or an engineered zinc-finger peptide. As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, “nucleic acid” is used to include any compound and/or substance that comprise a polymer of nucleotides. A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G). Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated.

Modifications can be introduced into a nucleotide sequence by standard techniques known in the art, such as site-directed mutagenesis and polymerase chain reaction (PCR)-mediated mutagenesis.

The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

These references are incorporated herein in their entirety.
1. Zhao, Y. Y. et al. Expanding RNA binding specificity and affinity of engineered PUF domains. Nucleic Acids Res. (2018). doi: 10.1093/nar/gkyl34
2. Konermann, S. et al. Transcriptome Engineering with RNA-Targeting Type VI-D CRISPR Effectors. Cell 173, 665-676.e14 (2018).
3. De Franco, S. et al. Exploring the suitability of RanBP2-type Zinc Fingers for RNA-binding protein design. Sci. Rep. 9, 1-13 (2019).
4. Dominguez, D. et al. Sequence, Structure, and Context Preferences of Human RNA Binding Proteins. Mol. Cell 70, 854-867.e9 (2018).
5. Wu, X. & Bartel, D. P. KpLogo: Positional k-mer analysis reveals hidden specificity in biological sequences. Nucleic Acids Res. (2017). doi: 10.1093/nar/gkx323
6. Loughlin, F. E. et al. The zinc fingers of the SR-like protein ZRANB2 are single-stranded RNA-binding domains that recognize 5′ splice site-like sequences. Proc. Natl. Acad. Sci. (2009)
7. Dominguez, D. et al. Sequence, Structure, and Context Preferences of Human RNA Binding Proteins. Mol. Cell 70, 854-867.e9 (2018)
8. Wu, X. & Bartel, D. P. KpLogo: Positional k-mer analysis reveals hidden specificity in biological sequences. Nucleic Acids Res. (2017)
9. Roe, D. R. & Thomas E. Cheatham, I. PTRAJ and CPPTRAJ: Software for Processing and Analysis of Molecular Dynamics Trajectory Data. J. Chem. Theory Comput. 9, 3084-3095 (2013)

### EXAMPLES

### Example 1—Engineering Sequence Specificity in an RNA-Binding Zinc Finger Protein

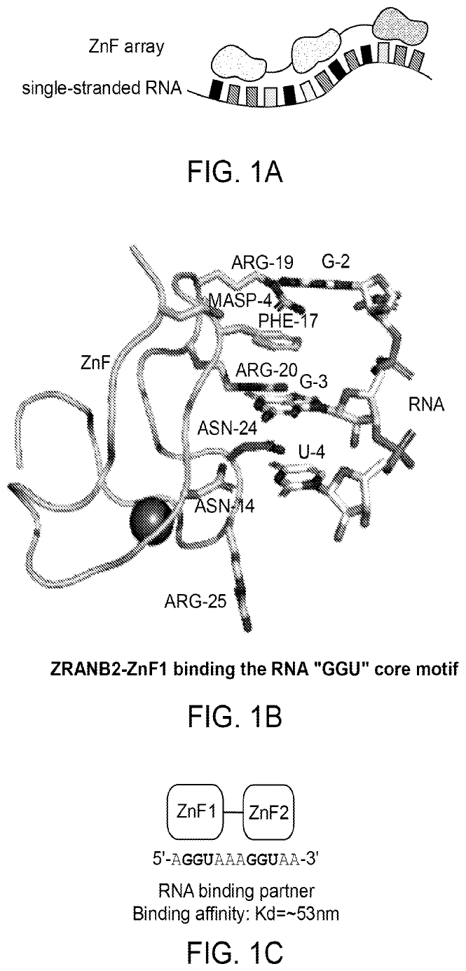

FIGS. 1A-1C show the structure of an exemplary zinc finger (ZRANB2-ZnF1) and interactions with an exemplary binding partner.

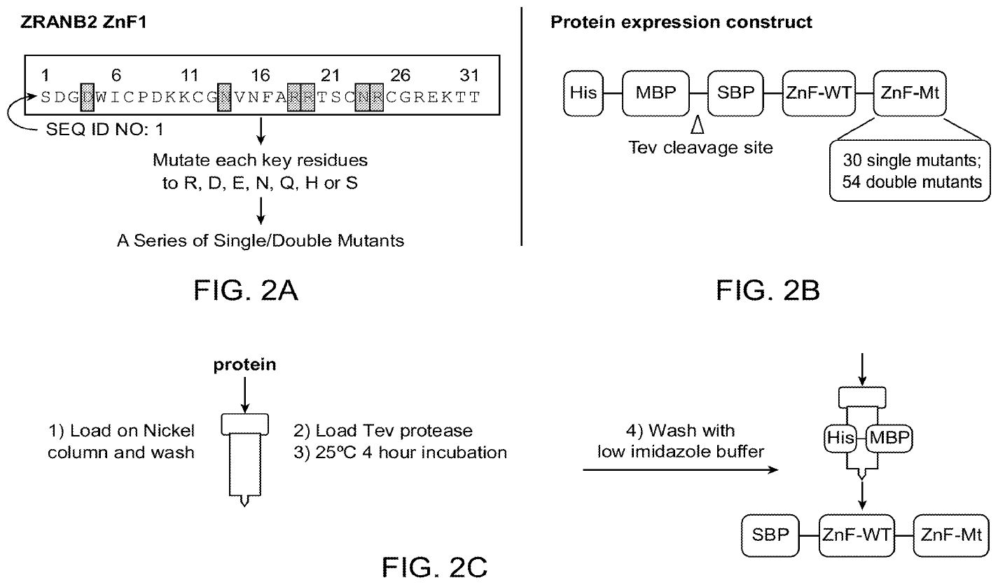

In this example, 30 single and 54 double mutations were systematically introduced at six key RNA-recognizing residues of the zinc finger Ran-binding domain-containing protein 2 (ZRANB2 ZnF) (FIGS. 2A-2B) and mutant protein was purified to result in mutant protein construct containing a streptavidin binding protein motif connected to a zinc finger wild-type motif, connected to zinc-finger mutant motif.

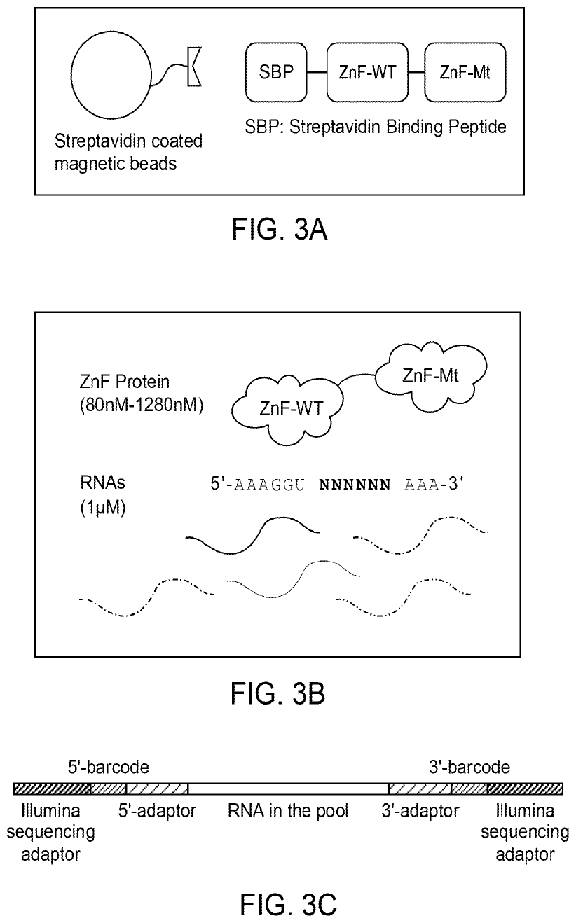

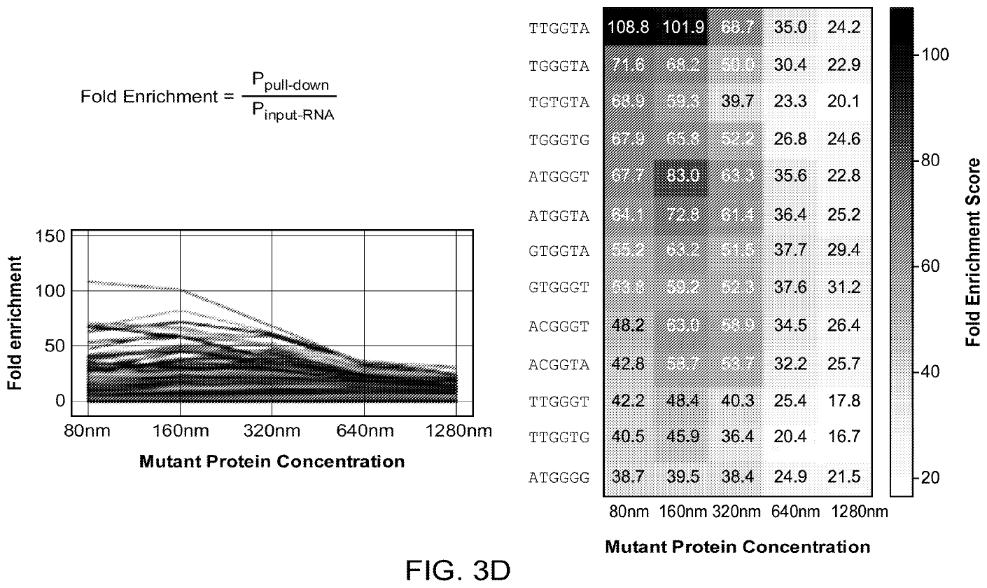

The RNA binding profile (motif preference, specificity and affinity) of each mutant ZnF was characterized using a modified RNAbind-n-seq (RBNS) assay (FIGS. 3A-3D). (See, for example, Dominguez, D. et al. Sequence, Structure, and Context Preferences of Human RNA Binding Proteins. Mol. Cell 70, 854-867.e9 (2018)). Briefly, mutant protein constructs were immobilized on streptavidin coated magnetic beads (FIG. 3A). Immobilized mutant protein constructs of various concentrations (80 nM, 160 nM, 320 nM, 640 nM, and 1280 nM) were incubated with a pool of random RNAs (FIG. 3B). Bound RNAs were pulled down using the magnetic bead and prepared for deep-sequencing by preparing the deep-sequencing Illumina construct shown in FIG. 3C. The fold-enrichment was determined by dividing the frequency of RNAs in the pull-down fraction to the frequency of RNAs in the input RNA. The overall RNA fold-enrichment for zinc fingers over multiple concentrations is shown in the left paned of FIG. 3D. The right panel of FIG. 3D shows top enriched RNA sequences.

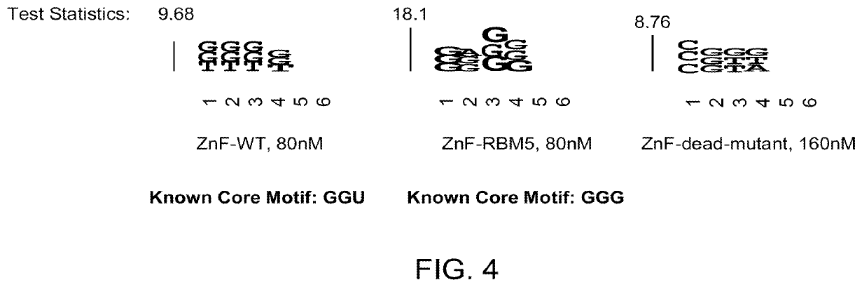

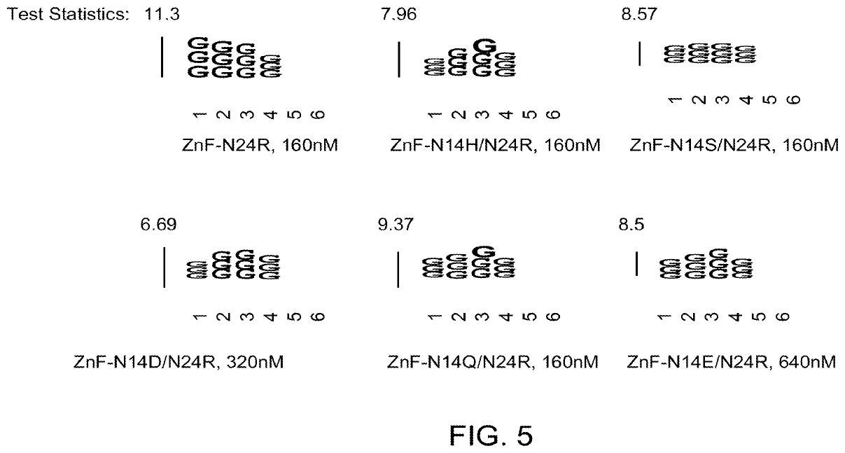

The core motif was identified using a kpLogo analysis. (See, for example, Wu, X. & Bartel, D. P. KpLogo: Positional k-mer analysis reveals hidden specificity in biological sequences. Nucleic Acids Res. (2017)). One mutant (ZnF-RBM5) with an altered sequence specificity, preferring GGG motif instead of GGU motif, was identified (FIG. 4). Additional mutants with altered binding specificity were also identified (FIG. 5)

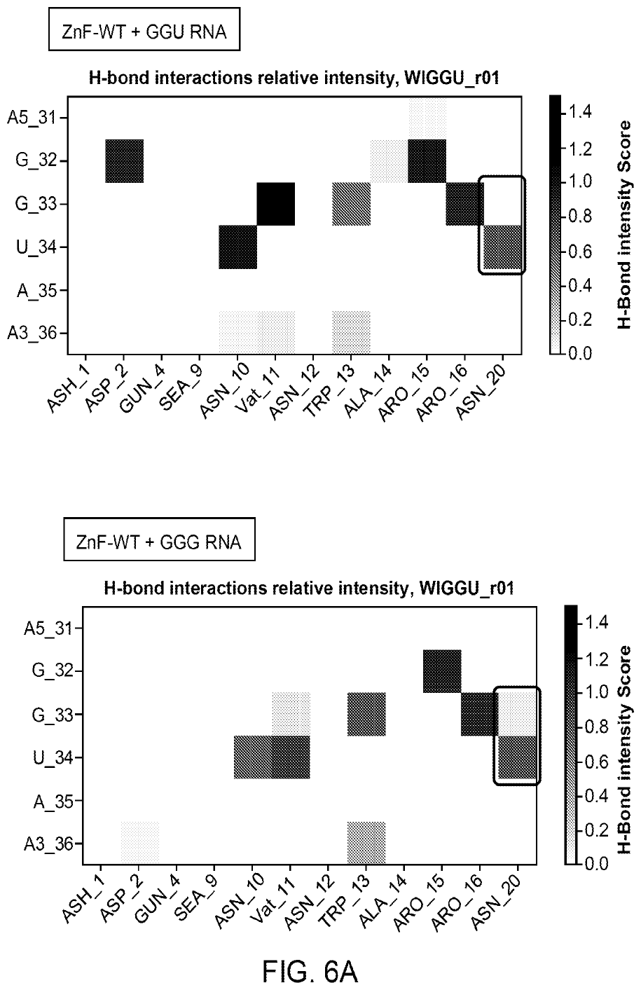

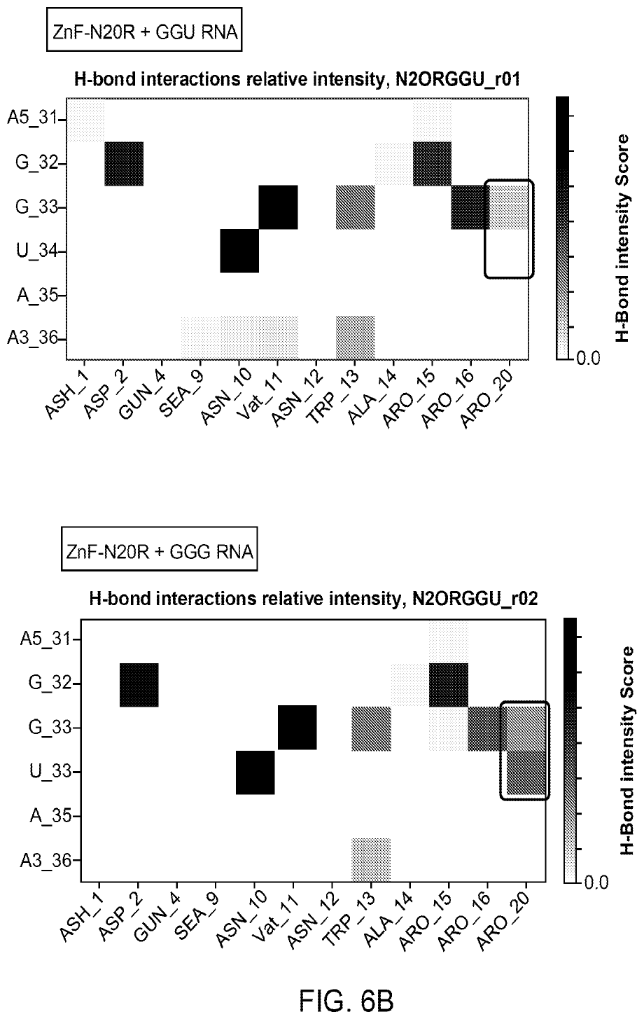

Complementing these in vitro assays, a series of 50-nanosecond all-atom molecular dynamics (MD) simulations were performed to investigate the mutant proteins' altered RNA binding preference. Three replicates of 50 ns NPT simulations for each condition were performed based on PDB structure 3G9Y, where N20 in 3G9Y correspond to N24 in ZRANB2 ZnF1. H-bond intensity scores were calculated for a unique pair of protein residue and RNA base (from CPPTRAJ4 H-bond statistics) and plotted on heatmaps (FIGS. 6A-6B). The mutated residue ARG did not maintain the H-bond interaction with U_34 when the RNA was “GGU”, compared to the WT residue ASN. In comparison, the mutated residue ARG maintained the H-bond interaction with the G_34 when the RNA was “GGG”.

The analysis of ZRANB2-RNA interactions both in vitro and in silico served as a foundation for RNA-binding ZnF designer protein engineering.

### Example 2—Exemplary Treatment of ALS and/or FTD in Patients

A patient presents symptoms and is diagnosed with ALS and/or FTD. The patient in need thereof is administered a therapeutically effective amount of any of the zinc finger peptides or any of the zinc finger peptide arrays described herein, wherein a zinc finger peptide includes a preferred RNA binding motif that specifically binds to a GGG motif of the mutant RNA expansion in the subject. The therapeutically effective amount of the pharmaceutical composition described herein reduces the incidence and/or severity of, stabilizes one or more characteristics of, and/or delays onset of, one or more symptoms of the ALS and/or FTD.

1. A zinc finger peptide comprising an amino acid sequence having at least 90% identity to SEQ ID NO: 1, wherein the zinc finger peptide comprises at least one amino acid substitution compared to SEQ ID NO: 1.

2. The zinc finger peptide of claim 1, wherein the zinc finger peptide is an engineered zinc finger peptide.

3. The zinc finger peptide of claim 1, wherein the zinc finger peptide has:
i) a substitution of N24R compared to SEQ ID NO: 1, and/or
ii) a substitution of N24H compared to SEQ ID NO: 1, and/or
iii) substitutions of N14D and N24R compared to SEQ ID NO: 1, and/or
iv) substitutions of N14D and N24H compared to SEQ ID NO: 1, and/or
v) substitutions of N14R and N24R compared to SEQ ID NO: 1, and/or
vi) substitutions of N14R and N24H compared to SEQ ID NO: 1, and/or
vii) substitutions of N14H and N24R compared to SEQ ID NO: 1, and/or
viii) substitutions of N14H and N24H compared to SEQ ID NO: 1, and/or
ix) substitutions of N14Q and N24R compared to SEQ ID NO: 1, and/or
x) substitutions of N14Q and N24H compared to SEQ ID NO: 1, and/or
xi) substitutions of N14E and N24R compared to SEQ ID NO: 1, and/or
xii) substitutions of N14S and N24R compared to SEQ ID NO: 1, and/or
xiii) substitutions of N14E and N24H compared to SEQ ID NO: 1.

4. The zinc finger peptide of claim 1, wherein the zinc finger peptide binds to RNA.

5. The zinc finger peptide of claim 1, wherein the zinc finger peptide has a preferred RNA binding motif that is not a GGU RNA sequence.

6. The zinc finger peptide of claim 1, wherein the zinc finger peptide has a preferred RNA binding motif that is a GGG RNA sequence.

7. The zinc finger peptide of claim 6, wherein the preferred RNA binding motif is determined with a fluorescent polarization assay or an electrophoretic mobility shift assay.

8. A zinc finger peptide array comprises:
a) between 2-30 zinc finger peptides of claim 1, and
b) a nucleotide spacer between the zinc finger peptides of (a).

9. The zinc finger peptide array of claim 8, wherein the zinc finger peptide array comprises 6 zinc finger peptides.

10. The zinc finger peptide array of claim 8, wherein the nucleotide spacer is between 1 and 10 nucleotides in length.

11. The zinc finger peptide array of claim 8, wherein the engineered zinc finger peptide binds to RNA.

12. The zinc finger peptide array of claim 8, wherein the engineered zinc finger peptide has a preferred RNA binding motif that is not a GGU RNA sequence.

13. A method of targeting nucleic acid sequences, the method comprising exposing an RNA sample to the zinc finger peptide array of claim 8
b) .

14. The method of claim 13, wherein the zinc finger peptide array comprises 6 zinc finger peptides.

15. The method of claim 13 or claim 14, wherein the nucleotide spacer is between 1 and 10 nucleotides in length.

16. The method of claim 13, wherein the engineered zinc finger peptide binds to RNA.

17. The method of claim 13, wherein the engineered zinc finger peptide has a preferred RNA binding motif that is not a GGU RNA sequence.

18. The method of claim 17, wherein the preferred RNA binding motif is determined with a fluorescent polarization assay or an electrophoretic mobility shift assay.

19. The method of claim 13, wherein the zinc finger peptide array is fused to an effector protein.

20. The method of claim 19, wherein the effector protein facilitates a function selected from nucleic acid degradation, nucleic acid splicing control, and nucleic acid translation.

21. A method of treating a G4-associated disease in a patient in need thereof, the method comprising administering a therapeutically effective amount of the zinc finger peptides of claim 1.

22. The method of claim 21, wherein the G4-associated disease is selected from the group consisting of amyotrophic lateral sclerosis (ALS) and frontotemporal dementia (FTD).
